(19) United States
(12) Patent Application Publication
(10) Pub. No.: US 2006/0287206 A1
(43) Pub. Date: Dec. 21, 2006

(54) Detergent bar and process for manufacture

(76) Inventors: Suresh Murigeppa Nadakatti, Bangalore (IN); Vijay Naik, Bangalore (IN)

Correspondence Address: UNILEVER INTELLECTUAL PROPERTY GROUP, 700 SYLVAN AVENUE,, BLDG C2 SOUTH, ENGLEWOOD CLIFFS, NJ, 07632-3100, US

(21) Appl. No.: 10/569,937

(22) PCT Filed: Jul. 30, 2004
(86) PCT No.: PCT/EP04/08658

§ 371 Date: Feb. 27, 2006

(30) Foreign Application Priority Data
Aug. 27, 2003 (IN) 0849/MUM/03

(51) Int. Cl. A61K 8/02

(52) U.S. Cl. 510/141

(57) ABSTRACT

A non-shrinking, melt cast solid cleansing composition free of soap is provided comprising i. 15-50% by weight of fatty acid selected from myristic acid, stearic acid, palmitic acid, hydroxy stearic acid, and mixtures thereof; ii. 2-40% by weight non soap detergent active iii. 30-60% water iv. optionally other ingredients such as functional actives and wherein the said composition is free of pure lyotropic liquid crystalline phase in the temperature range 20-100° C. and forms an isotropic liquid phase or a dispersion of lyotropic liquid crystalline phase in the continuum of isotropic liquid in the temperature range 40-100° C.

### TECHNICAL FIELD

The present invention relates to a melt cast solid cleansing composition with a neutral/skin/mildly acidic pH comprising high levels of liquid benefit agents/water, that would be rigid and does not shrink on storage. Preferably the solid cleansing composition is a detergent bar.

### BACKGROUND AND PRIOR ART

Solid cleansing compositions, for example in the form of detergent bars, are generally prepared by either melt cast route or by extrusion. The compositions comprise detergent molecules that may be soap or non-soap actives or a combination of the two along with other conventional ingredients. The compositions are generally alkaline with a pH of about 9-10 and above.

The skin has a pH in the range 5.5-6.5 and high pH cleansing compositions are irritants to the skin. It is generally known that cleansing compositions with neutral/skin/mildly acidic pH result in lower skin irritation (cf. Report in International Journal of Dermatology 2002, 41, 494-499). Attempts have been made to formulate solid cleansing compositions that have low pH which match the pH of the skin or are neutral and thus reduce the irritation to the skin.

In the manufacture of solid cleansing compositions the process of casting has a specific advantage over the process of extrusion as it enables one to manufacture bar compositions comprising high levels of water, air, and liquid benefit agents to obtain low cost yet high performing bars. Increasing the liquid content in bars generally helps in improved in-use properties and functional benefits such as superior quality of lather, superior perfume impact, superior delivery of functional ingredients, etc. One of the key problems of bars with high moisture content is that on storage they tend to lose water and shrink.

U.S. Pat. No. 5,262,079 (P&G, 1993), discloses firm, ultra mild, low smear, neutral pH carboxylic acid based cleansing bars that contain high level of moisture and synthetic surfactants. The bar comprises mixture of free and neutralised monocarboxylic acids, bar firmness aid and water. The level of neutralised carboxylic acid is 20-65% of the mixture of the said free and neutralised monocarboxylic acid. The level of water in the said bar compositions is from about 15-55% by weight of the said bar.

U.S. Pat. No. 5,227,086 (P&G, 1993) mentions that the bar smear is especially poor in neutral pH bars that contain high levels of synthetic surfactants and discloses ultra mild, weakly acidic skin pH carboxylic acid based bars. The bar comprises free monocarboxylic acid or mixture of free and neutralised monocarboxylic acids, bar firmness aid and water. The level of neutralised carboxylic acid in the composition is 0-15% of the mixture of the said free and neutralised monocarboxylic acid. The level of water in the said bar compositions is from about 15-55% by weight of the said bar.

In the above prior art the level of neutralised monocarboxylic acid controls the pH of the composition. In U.S. Pat. No. 5,262,079 it is essential to have neutralised monocarboxylic acid to obtain neutral pH bars. In the absence of the neutralised carboxylic acids in the composition as disclosed in U.S. Pat. No. 5,227,086 the pH is about 4-6.5. Also, neutralised monocarboxylic acids, which are used to control pH of such bar compositions, are generally harsher on skin and its use affects the mildness properties of the bar

It has now been observed that the presence or absence of neutralized monocarboxylic acid while controlling the pH is not necessarily solely responsible for desired rigidity of a melt cast bar and given the need for rigid bars in the art there is a continuing need to provide cast bar compositions having good rigidity properties apart for being mild on skin. In particular, need exists in the art to prepare non-shrinking and rigid bars with high levels of liquid actives, liquid functional ingredients, and water having good skin-feel properties.

It is thus the basic object of the present invention to selectively provide non soap rigid bars at high levels of liquid actives, liquid functional ingredients, and water.

Another object of the present invention is to provide solid cleansing compositions comprising high levels of liquid benefit agents and water that do not shrink on storage.

Yet another object of the present invention is to provide rigid, non-shrinking, neutral/skin/mildly acidic pH solid cleansing compositions with high levels of liquid actives and water which would be mild on skin.

Another object of the present invention is to avoid the use of neutralised monocarboxylic acids in solid cleansing bar compositions and yet obtain rigid bars of controlled pH and in particular, desired neutral/skin/mildly acidic pH.

Another object is directed to a melt-cast process for the manufacture of rigid, non-shrinking, neutral/skin/mildly acidic pH solid cleansing compositions with high levels of liquid actives and water which would be mild on skin.

### SUMMARY OF THE INVENTION

The present invention provides a melt cast solid cleansing composition free of soap comprising
i. 15-50% by weight of fatty acid selected from myristic acid, stearic acid, palmitic acid, hydroxy stearic acid, and mixtures thereof;
ii. 2-40% by weight non soap detergent active
iii. 30-60% water
iv. optionally other ingredients such as functional actives and
   wherein the said composition is free of pure lyotropic liquid crystalline phase in the temperature range 20-100° C. and forms an isotropic liquid phase or a dispersion of lyotropic liquid crystalline phase in the continuum of isotropic liquid in the temperature range 40-100° C.

Preferably the solid cleansing composition is in the form of a detergent bar

It is particularly preferred that the pH of the solid cleansing composition is between 5.5-8.5.

It has now been possible by way of the above disclosed selective compositions to provide rigid, non-shrinking, neutral/skin/mildly acid pH solid cleansing compositions containing high levels of liquid actives, liquid functional ingredients, and water. Importantly, the compositions of the invention involves a selective phase behavior dependent formulation based on selective combination of (i) fatty acids preferably free monocarboxylic acids selected from myristic, palmitic, stearic or hydroxystearic acid and mixtures thereof as the structurant, and (ii) the non-soap detergent active of a selective pH.

In accordance with another aspect of the present invention there is provided a process for providing a melt cast solid cleansing composition, the method comprising the steps of:
i. screening and providing a detergent composition comprising 15-50% by wt. of fatty acid selected from myristic acid, stearic acid, palmitic acid, hydroxy stearic acid, and mixtures thereof, 2-40% by wt. of non-soap detergent active, 30-60% by wt. water and optional functional ingredients and which would be free of pure lyotropic liquid crystalline phase in the temperature range 20-100° C. and form an isotropic liquid phase or a dispersion of lyotropic liquid crystalline phase in the continuum of isotropic liquid in the temperature range 40-100° C.
ii. heating the above detergent composition in the temperature of 70-95° C.
iii. pouring the melt of the above detergent composition into a suitable mould and cooling.

### DETAILED DESCRIPTION OF THE INVENTION

The present invention relates to a melt cast solid cleansing composition with a neutral/skin/mildly acidic pH comprising high levels of liquid benefit agents/water, that does not shrink on storage. The composition comprises particular fatty acids, non-soap detergent active, water, and optional ingredients such as functional actives. The solid cleansing composition is characterised in that the said composition is free of pure lyotropic liquid crystalline phase in the temperature range 20-100° C. and forms an isotropic liquid phase or a dispersion of lyotropic liquid crystalline phase in the continuum of isotropic liquid in the temperature range 40-100° C.

The presence of isotropic liquid phase and the liquid crystalline phases in surfactant systems is preferably detected using optical polarising microscopy technique. Liquid crystalline phases are inherently anisotropic, making the index of refraction depend on orientation. In general, such birefringent systems change the plane of polarisation of polarized light. An isotropic liquid appears black between crossed polarizers, but a liquid crystalline system is more transparent e.g. lamellar liquid crystalline phases show maltese-cross or oil streak (large loop like) textures whereas hexagonal phases show non geometric, fan like textures. Several textures may be observed within the same phase structure. Characteristic textures of various lyotropic liquid crystalline phases (and their dispersions) have been reported in following references: (i) “The Aqueous Phase Behaviour of Surfactants” by Robert G. Laughlin, Academic Press, New York, 1994, Pages 538-542. (ii) “The Colloidal Domain Where Physics, Chemistry, Biology, and Technology Meet” by D. Fennell Evans, Hakan Wennerstrom, VCH Publishers, New York, 1994, Pages 251-252.

Fatty Acid:

The saturated fatty acid is selected from myristic acid, stearic acid, palmitic acid, hydroxystearic acid and mixtures thereof. The saturated fatty acid in the composition is from 15-50% by weight.

Detergent Active:

The solid cleansing compositions according to the invention essentially comprise detergent actives that are non-soap based. It is preferable to employ non-soap detergent actives that are selected from anionic, non-ionic, cationic, amphoteric or zwitterionic surfactants or their mixtures.

Anionic Surfactants:

Suitable anionic detergent active compounds are water soluble salts of organic sulphuric reaction products having in the molecular structure an alkyl radical containing from 8 to 22 carbon atoms, and a radical chosen from sulphonic acid or sulphuric acid ester radicals and mixtures thereof. Some examples of synthetic anionic detergent active compounds are linear alkyl benzene sulphonate, sodium lauryl sulphate, sodium lauryl ether sulphate, alpha olefin sulphonate, alkyl ether sulphate, fatty methyl ester sulphonate, alkyl isothionate, and the like.

The cations most suitable in above detergent active species are sodium, potassium, ammonium, and various amines e.g. monoethanolamine, diethanolamine and triethanolamine.

Nonionic Surfactants:

Suitable non-ionic detergent active compounds can be broadly described as compounds produced by the condensation of alkylene oxide groups, which are hydrophilic in nature, with an organic hydrophobic compound which may be aliphatic or alkyl aromatic in nature. The common non-ionic surfactants are the condensation products of aliphatic alcohols having from 8 to 22 carbon atoms in either straight or branched chain configuration with ethylene oxide, such as a coconut oil ethylene oxide condensate having from 2 to 15 moles of ethylene oxide per mole of coconut alcohol. Some examples of non-ionic surfactants are alkyl phenol ethylene oxide (EO) condensate, tallow alcohol 10 EO condensate, alkyl de-methyl amine oxides, lauryl mono-ethanolamide, sugar esters, and the like

Other Surfactants:

Some examples of amphoteric detergent active are coco amidopropyl betaine, cocobetaine, and the like.

It is also possible optionally to include cationic or zwitterionic detergent actives in the compositions according to the invention.

Further examples of suitable detergent-active species are given in the following reference: “Handbook of Surfactants”, M. R. Porter, Chapman and Hall, New York, 1991.

The detergent active to be employed in the solid cleansing composition of this invention is preferably anionic and will generally be present at a level of up to 40%.

Liquid Benefit Agents:

According to a preferred aspect of the invention, liquid benefit materials such as moisturisers, emollients, fabric conditioners, etc. are incorporated in the composition. Examples of moisturisers and humectants include silicone oil, polyols, glycerol, cetyl alcohol, carbopol 934, ethoxylated castor oil, paraffin oils, lanolin and its derivatives.

Optional Ingredients:

Other optional ingredients such as salting-in-electrolytes, polyols, fillers, colour, perfume, opacifier, preservatives, one or more water insoluble particulate materials such as talc, kaolin, polysaccharides and other conventional ingredients may be incorporated in the composition.

Process:

A melt of the detergent composition comprising 15-50% by weight of saturated fatty acid, 2-40% by weight of non-soap detergent active, 30-60% water, and optional functional liquid ingredients is prepared at about 70 to 95 deg. C. This melt is poured into any suitable mould or a pre-formed polymeric mould. The compositions where hydroxystearic acid is used as the fatty acid structurant the system was first cooled under quiescent conditions in a hot oven maintained at about 55 deg. C. for about 45 minutes and then further cooled under ambient conditions to obtain a rigid tablet. The compositions where myristic acid or palmitic acid or stearic acid or mixtures of these fatty acids were used as fatty acid structurants, the systems were cooled directly under ambient conditions (about 25 deg. C.) to obtain a rigid tablet. For these compositions the step of holding the compositions at 55 deg. C. was not present.

The above composition is characterised in that the said composition does not show pure lyotropic liquid crystalline phase in the temperature range 20-100° C. and forms an isotropic liquid phase or a dispersion of lyotropic liquid crystalline phase in the continuum of isotropic liquid in the temperature range 40-100° C.

The mould may be suitably selected to produce near net shape tablet or to produce bars/blocks. The bars/blocks may be further shaped in to detergent article.

The solid cleansing compositions according to the invention are rigid enough to be conveniently held in hand, economical, and exhibit good in-use properties. The preferred compositions exhibit yield stress values greater than 75 kPa as measured using the automatic penetrometer.

If the solid detergent article is produced using a near net shape thermoformed polymer, the mould is sealed to obtain a cast-in pack detergent composition. To obtain cast-in pack detergent composition the mould is preferably sealed immediately after filling the mould.

The composition can be prepared in bar form using a continuous process comprising steps of:
i. filling a continuous tube of flexible material formed online and sealed at the bottom end, with a melt of the castable composition, where the tube acts as a sleeve to the composition, and simultaneously conveying through a cross section constraining guide to achieve desired area of cross section of the filled sleeve that is independent of the perimeter
ii. sealing the filling end of the filled tubular sleeve without air entrapment to obtain a cast-in-sleeve melt
iii. solidifying and simultaneously shaping the said melt by cooling the said filled sleeve on a suitable mould to obtain a cast-in-sleeve log
iv. cutting the said shaped and solidified cast composition into billets/tablets
v. optionally flow wrapping the said logs/billets/tablets

The bars may be dehydrated after demoulding to obtain aerated, low density bars.

The invention will now be illustrated with respect to the following non-limiting examples.

### EXAMPLE 1

### Screening of the Compositions with Respect to Their Phase Behaviour

The composition according to the invention is characterised in that the said composition does not show pure lyotropic liquid crystalline phase in the temperature range 20-100° C. and forms an isotropic liquid phase or a dispersion of lyotropic liquid crystalline phase in the continuum of isotropic liquid in the temperature range 40-100° C. The comparative examples according to the invention and beyond the invention are presented in Table 1.

i. Phase Characterisation:

Phase characterisation of the composition was carried out using optical polarising microscopy technique. A drop of the melt of the composition at elevated temperature of about 90° C. was transferred on to a microscope glass slide and covered with a cover slip. The edges of the cover slip were sealed using UV glue to minimise moisture loss. The glass slide was mounted on the stage of the microscope provided with a controlled heating/cooling facility. The system was heated to 95° C. at a rate of 1° C. per minute. The temperature at which isotropic liquid phase or its dispersion, or pure liquid crystalline phase was formed, was noted down. The system was then cooled to bring about solidification at the rate of 1° C. per minute to check if pure liquid crystalline phases are formed during cooling. Lyotropic liquid crystalline phases are inherently anisotropic, making the index of refraction depend on orientation. In general, such birefringent systems change the plane of polarisation of polarized light. An isotropic liquid appears black between crossed polarizers, but a liquid crystalline system is more transparent e.g. lamellar liquid crystalline phases show maltese-cross or oil streak (large loop like) textures whereas hexagonal phases show non geometric, fan like textures. Several textures may be observed within the same phase structure. Characteristic textures of various lyotropic liquid crystalline phases (and their dispersions) have been reported in following references: (i) “The Aqueous Phase Behaviour of Surfactants” by Robert G. Laughlin, Academic Press, New York, 1994, Pages 538-542. (ii) “The Colloidal Domain Where Physics, Chemistry, Biology, and Technology Meet” by D. Fennell Evans, Hakan Wennerstrom, VCH Publishers, New York, 1994, Pages 251-252.

ii. Preparation of Detergent Tablet

The phase behaviour of the compositions as presented in Table 1 was determined using optical polarising microscopy using the procedure described above. A melt of the detergent composition as presented in Table 1, at an elevated temperature of about 90° C. is poured in to a rectangular stainless steel mould of dimensions 75 mm (L)×55 mm (W)×40 mm (H). The composition was allowed to cool in a hot oven maintained at 55° C. for about 45 minutes. The mould was then cooled under ambient conditions (about 25 deg. C.) to obtain a rigid detergent tablet. The yield stress was measured using the procedure described below and is presented in table 1.

The compositions shown in the other tables (tables 2 to 6) where the fatty acid used is myristic acid or palmitic acid or stearic acid or mixtures of these fatty acids were filled into moulds between 70 to 90 deg. C. and cooled under ambient conditions (about 25 deg. C.) to bring about solidification. For these compositions the step of holding the systems at 55 deg. C. in a hot oven before further cooling down to ambient conditions (which was followed for compositions wherein hydroxystearic acid was used as the fatty acid in the composition) was not present.

iii. Yield Stress Measurement:

The detergent tablets were then kept in oven maintained at 25° C. for 4 hours and allowed to equilibrate. The yield stress of the tablets at 25° C. was measured using a automatic penetrometer using the procedure described below.

The automatic penetrometer used for yield stress measurements was model PNR 10 from M/s Petrotest Instruments GmbH. Standard Hollow Cone (part # 18-0101, as per ASTM D 217-IP 50) along with Plunger (part # 18-0042) was used for the measurements. The cone consisted of a conical body of brass with detachable, hardened steel tip. The total mass of the cone was 102.5 g. The total mass of the movable plunger was 47.5 g. Total mass of cone and plunger that fall on the detergent tablet was therefore 150 g. Additional weights of 50 g and 100 g (making the total weight falling on the sample 200 g and 250 g, respectively) were also used. The yield stress values of the sample at 25° C. were measured using the standard procedure comprising following steps:

The detergent tablet was placed on the table of the penetrometer.

The measuring device of the penetrometer was lowered so that the tip of the penetrometer touched the tablet but did not penetrate it.

The measurement operation was started by pressing “start” key.

The penetration depth was read in mm as indicated on the display.

The measured penetration depth value was used to calculate the yield stress of the detergent tablet using the following equation:

$$\begin{matrix}Yield stress = Applied force / ( Projected area of the cone ) \\ = ( m ⨯ g ) ⨯ {10}^{3} / [ {π ( p tan \frac{1}{2} θ + \frac{1}{2} tip diameter )}^{2} ]\end{matrix}$$

where
Yield stress is in kPa
m: total mass falling on the flat surface of the bar in kg
g: acceleration due to gravity in m/s²
p: penetration achieved in mm
θ: Cone angle (30°)
tip diameter=0.359 mm

According to the above equation if the measured penetration depth is <10 mm for 200 g total mass falling on the sample then the yield stress of the detergent tablet is >75 kPa. The penetration values reported in Tables 1-6 are for 200 g total mass falling on the detergent tablet.

iv) Shrinkage Studies

The dimensions of the rectangular shaped tablets prepared using the procedure described above were measured using a vernier callipers. The tablets were kept open (unpacked) in the laboratory (about 25 deg. C. and relative humidity about 50%) for several days and allowed to lose moisture. Typically the tablets lost 10% to 40% of weight after dehydration. The dimensions of the dehydrated tablet were measured. The % volume shrinkage was determined from the initial and final volume of the rectangular shaped detergent tablet. The detergent tablets were considered to be non-shrinking if the % volume shrinkage is less than 10%. The non-shrinking detergent bar compositions result in aerated, low density bars upon dehydration.

v) Measurement of pH

A solution of 1% (by weight) of the composition in distilled water was prepared. The pH of the solution was measured using a calibrated pH meter.

TABLE 1

| Components (% w/w) | Ex 1 | Ex A | Ex B |
|---|---|---|---|
| Hydroxy stearic acid (HSA) | 25 | 25 | 15 |
| Sodium lauryl ether sulphate | 30 | 40 | 30 |
| (SLES) | | | |
| Water | 36 | 26 | 46 |
| Poly ethylene glycol (PEG) 1500 | 9 | 9 | 9 |
| Does the composition show pure | No | Yes. H1 | Yes. H1 |
| liquid crystalline phase in the | | phase* | phase* |
| temperature range | | | |
| 20-100° C.? | | | |
| Does the composition form | Yes | No | No |
| isotropic liquid phase or a | | | |
| dispersion of lyotropic | | | |
| liquid crystalline phase in | | | |
| the continuum of isotropic | | | |
| liquid in the temperature | | | |
| range 40-100° C.? | | | |
| Does the composition show | Yes | Yes | Yes |
| presence of solid crystallites | | | |
| upon cooling? | | | |
| Shrinkage (% v/v) | <10% | <10% | <10% |
| pH | 7.5 | 8.2 | 7.6 |
| Penetration (mm) | 7.6 | 8.4 | 15.2 |
| Yield stress @ 25° C. | 121 | 106 | 35 |
| (kPa) | | | |

*H1 is pure hexagonal liquid crystalline phase.

The data in Table 1 shows that only the composition as described in Ex 1 that satisfied the phase characterisation according to the invention formed rigid detergent tablet exhibiting yield stress of 121 kPa. The compositions of Examples A and B are soft because they form a pure lyotropic liquid crystalline phase in the temperature range 20-100° C. and do not form isotropic liquid phase or its dispersion in the temperature range 40-100° C. This demonstrates that it is essential to satisfy both the phase behaviour criteria to obtain bars that are rigid using compositions containing high levels of water and liquid detergent actives.

### EXAMPLE 2

### Effect of Fatty Acid Structurant on Shrinkage

Examples in Table 2 demonstrate that only bar compositions comprising high levels of water and liquid actives wherein the fatty acid structurant is myristic acid or stearic acid or palmitic acid or hydroxystearic acid or mixture of these acids, shrink less than 10% by volume upon dehydration/moisture loss. The bar composition shown in Ex C where lauric acid is used as the fatty acid structurant shrinks >10% upon moisture loss.

TABLE 2

| Composition | | | | | | |
|---|---|---|---|---|---|---|
| (% w/w) | Ex 2.1* | Ex 2.2* | Ex 2.3* | Ex 2.4* | Ex 2.5* | Ex C* |
| Stearic acid | 30 | | | | | |
| Palmitic acid | | 30 | | | 20 | |
| Hydroxy- | | | 30 | | | |
| stearic | | | | | | |
| acid | | | | | | |
| Myristic acid | | | | 30 | | |
| Lauric acid | | | | | | 30 |
| DEFI** | 21 | 21 | 21 | 21 | 21 | 21 |
| Water | 39 | 39 | 39 | 39 | 39 | 39 |
| Propylene- | 10 | 10 | 10 | 10 | 10 | 10 |
| glycol | | | | | | |
| Shrinkage | <10 | <10 | <10 | <10 | <10 | >10 |
| (% v/v) | | | | | | |
| pH | 6.7 | 6.6 | 7.0 | 8.0 | 8.1 | 6.3 |
| Penetration | 7.3 | 7.0 | 4.2 | 7.9 | 8.9 | >10 |
| (mm) | | | | | | |
| Yield stress | 138 | 150 | 371 | 119 | 94 | <75 |
| @ 25° C. | | | | | | |
| (kPa) | | | | | | |

*All compositions satisfy the phase behaviour criterion
**DEFI: 73% sodium coco isethionate, 20% free fatty acid, 7% water

### EXAMPLE 3

### Effect of Non-Soap Detergent on pH of the Composition

Examples 3.1 to 3.4 show that the pH of the composition is dependent on the non-soap detergent active used for a given level of fatty acid, water, and other functional liquid ingredient.

TABLE 3

| Composition (% w/w) | Ex 3.1* | Ex 3.2* | Ex 3.3* | Ex 3.4* | Ex 3.5* |
|---|---|---|---|---|---|
| Stearic acid | 30 | 30 | 30 | 30 | 30 |
| Sodium | 15 | | | | |
| cocoisethionate | | | | | |
| (SCI)** | | | | | |
| Sodium lauryl ether | | 15 | | | |
| sulphate (SLES) | | | | | |
| Alpha olefin | | | 15 | | |
| sulphonate (AOS) | | | | | |
| Disodium oleate | | | | 15 | |
| sulphosuccinate | | | | | |
| (OSS) | | | | | |
| Disodium oleate | | | | | 15 |
| sulphosuccinate | | | | | |
| (OSS) | | | | | |
| Water | 45 | 45 | 45 | 45 | 45 |
| PEG1500*** | 10 | 10 | 10 | 10 | 10 |
| Shrinkage (% v/v) | <10 | <10 | <10 | <10 | <10 |
| pH | 6.9 | 6.9 | 7.2 | 6.6 | 8.5 |
| Penetration (mm) | 5.3 | 8.2 | 5.9 | 6.2 | 9.6 |
| Yield stress | 244 | 113 | 202 | 186 | 82 |
| @ 25° C. (kPa) | | | | | |

*All compositions satisfy the phase behaviour criterion;
**Added as DEFI comprising 73% sodium coco isethionate, 20% free fatty acid, 7% water
***PEG1500: Polyethylene glycol 1500

### EXAMPLE 4

### Range of Fatty Acids that can be Used in the Composition

Examples 4.1 to 4.4 in Table 4 show that rigid bars containing high levels of water and liquid actives can be obtained using stearic acid in the compositions ranging from 25 to 40% by weight.

TABLE 4

| Composition (% w/w) | Ex 4.1* | Ex 4.2* | Ex 4.3* | Ex 4.4* |
|---|---|---|---|---|
| Stearic acid | 25 | 30 | 35 | 40 |
| DEFI** | 21 | 21 | 21 | 21 |
| Water | 44 | 39 | 34 | 29 |
| PEG1500*** | 10 | 10 | 10 | 10 |
| Shrinkage (% v/v) | <10 | <10 | <10 | <10 |
| pH | 7.3 | 6.8 | 7.1 | 6.6 |
| Penetration (mm) | 5.6 | 5.3 | 4.1 | 3.8 |
| Yield stress @ 25° C. (kPa) | 223 | 246 | 379 | 430 |

*All compositions satisfy the phase behaviour criterion
**DEFI comprises 73% sodium coco isethionate, 20% free fatty acid, 7% water
***PEG1500: Polyethylene glycol 1500

### EXAMPLE 5

### Range of Non-Soap Detergent Actives

Examples 5.1 to 5.3 shown in Table 5 demonstrate that rigid bars can be obtained using 30% palmitic acid as the structurant, 10% PEG 1500, and 15 to 30% sodium cocoisothionate as non-soap detergent active.

TABLE 5

| Composition (% w/w) | Ex 5.1* | Ex 5.2* | Ex 5.3* |
|---|---|---|---|
| Palmitic acid | 30 | 30 | 30 |
| DEFI** | 15 | 20 | 30 |
| Water | 45 | 40 | 30 |
| PEG1500*** | 10 | 10 | 10 |
| Shrinkage (% v/v) | <10 | <10 | <10 |
| pH | 8.0 | 7.7 | 7.0 |
| Penetration (mm) | 6.5 | 6.8 | 1.5 |
| Yield stress @ 25° C. (kPa) | 168 | 158 | >1000 |

*All compositions satisfy the phase behaviour criterion
**DEFI comprises 73% sodium coco isethionate, 20% free fatty acid, 7% water
***PEG1500: Polyethylene glycol 1500

### EXAMPLE 6

### Other Compositions

Examples 6.1 to 6.4 shown in Table 6 demonstrate that rigid, low pH, bar compositions comprising high levels of non-soap detergent active, and water can be prepared using fatty acid as structurant.

TABLE 6

| Composition (% w/w) | Ex 6.1* | Ex 6.2* | Ex 6.3* | Ex 6.4* |
|---|---|---|---|---|
| Hydroxystearic acid | 30 | 35 | 30 | |
| Stearic acid | | | | 30 |
| Sodium lauryl ether | 20 | 30 | | |
| sulphate (SLES) | | | | |
| DEFI** | | | 21 | 21 |
| Water | 50 | 35 | 49 | 49 |
| Shrinkage (% v/v) | <10 | <10 | <10 | <10 |
| pH | 7.9 | 7.8 | 7.2 | 5.5 |
| Penetration (mm) | 6.4 | 5.5 | 5.1 | 6.6 |
| Yield stress @ 25° C. (kPa) | 176 | 232 | 262 | 167 |

*All compositions satisfy the phase behaviour criterion
**DEFI comprises 73% sodium coco isethionate, 20% free fatty acid, 7% water

The above results demonstrate the selective phase behavior dependent composition of the invention involving the combination of fatty acid and non-soap detergent active of selective pH combination for desired rigidity of melt-cast cleansing solid composition and which provide the desired mildness and also would retain high levels liquid benefit agents/water without shrinking on storage.

The various features and embodiments of the present invention, referred to in individual sections above apply, as appropriate, to other sections, mutatis mutandis. Consequently features specified in one section may be combined with features specified in other sections, as appropriate.

All publications mentioned in the above specification are herein incorporated by reference. Various modifications and variations of the described methods and products of the invention will be apparent to those skilled in the art without departing from the scope of the invention. Although the invention has been described in connection with specific preferred embodiments, it should be understood that the invention as claimed should not be unduly limited to such specific embodiments. Indeed, various modifications of the described modes for carrying out the invention which are apparent to those skilled in the relevant fields are intended to be within the scope of the following claims.

1. A non-shrinking, melt cast solid cleansing composition free of soap consisting of
i. 15-50% by weight of fatty acid selected from myristic acid, stearic acid, palmitic acid, hydroxy stearic acid, and mixtures thereof;
ii. 2-40% by weight non soap detergent active
iii. 30-60% water; and
iv. optionally other ingredients selected from lliquid benefit materials, salting-in-electrolytes, polyols, fillers, colour, perfume, opacifiers, preservatives, and water insoluble particulate materials;
wherein the said composition is free of pure lyotropic liquid crystalline phase in the temperature range 20-100° C. and forms an isotropic liquid phase or a dispersion of lyotropic liquid crystalline phase in the continuum of isotropic liquid in the temperature range 40-100° C.

2. A non-shrinking, melt cast solid cleansing composition free of soap comprising
i. 15-50% by weight of fatty acid selected from myristic acid, palmitic acid, hydroxy stearic acid, and mixtures thereof;
ii. 2-40% by weight non soap detergent active
iii. 30-60% water; and
iv. optionally other ingredients such as functional actives;
wherein the said composition is free of pure lyotropic liquid crystalline phase in the temperature range 20-100° C. and forms an isotropic liquid phase or a dispersion of lyotropic liquid crystalline phase in the continuum of isotropic liquid in the temperature range 40-100° C.

3. A solid cleansing composition according to claim 1 wherein the pH of the composition is between 5.5 and 8.5.

4. A solid cleansing composition according to claim 1 in the form of a detergent bar.

5. A process for providing a melt cast solid cleaning composition comprising the steps of:
i. screening and providing a detergent composition consisting of 15-50% by wt. of fatty acid selected from myristic acid, stearic acid, palmitic acid, hydroxy stearic acid, and mixtures thereof; 2-40% by wt. of non soap detergent active, 30-60% by wt. water; and optional other ingredients selected from liquid benefit materials, salting-in-electrolytes, polyols, fillers, colour, perfume, opacifiers, preservatives, and water insoluble particulate materials and which would be free of pure lyotropic liquid crystalline phase in the temperature range 20-100° C. and form an isotropic liquid phase or a dispersion of lyotropic liquid crystalline phase in the continuum of isotropic liquid in the temperature range 40-100° C.;
ii. heating the above detergent composition in the temperature of 70-95° C.;
iii. pouring the melt of the above detergent composition into a suitable mould and cooling.

6. A process for providing a melt cast solid cleansing composition comprising the steps of:
i. screening and providing a detergent composition comprising 15-50% by wt. of fatty acid selected from myristic acid, palmitic acid, hydroxyl stearic acid, and mixtures thereof, 2-40% by wt. of non-soap detergent active, 30-60% by wt. water and optional functional ingredients and which would be free of pure lyotropic liquid crystalline phase in the temperature range 20-100° C. and form an isotropic liquid phase or a dispersion of lyotropic liquid crystalline phase in the continuum of isotropic liquid in the temperature range 40-100° C.
ii. heating the above detergent composition in the temperature of 70-95° C.;
iii. pouring the melt of the above detergent composition into a suitable mould and cooling.
